(19) United States
(12) Patent Application Publication
(10) Pub. No.: US 2011/0250128 A1
(43) Pub. Date: Oct. 13, 2011

(54) Method for tissue characterization based on beta radiation and coincident Cherenkov radiation of a radiotracer

(76) Inventors: Colin M Carpenter, Redwood City, CA (US); Lei Xing, Palo Alto, CA (US); Conroy Ghin Chee Sun, Sunnyvale, CA (US); Guillem Pratx, Moutain View, CA (US)

(21) Appl. No.: 13/066,351

(22) Filed: Apr. 12, 2011

Related U.S. Application Data
(60) Provisional application No. 61/342,321, filed on Apr. 12, 2010.

(51) Int. Cl. A61K 51/00; C12Q 1/02

(52) U.S. Cl. 424/1.11; 435/29

(57) ABSTRACT

A method of characterizing a tissue sample is provided that includes injecting a tissue sample with radiotracers, where the radiotracers include beta-emitter radio tracers, the beta-emitter radio tracers emit beta particles according to a decay of the beta-emitter radio tracers, and measuring the beta particles or Cherenkov radiation from the beta particles in the tissue sample, and determining a condition of the radio tracers in the tissue sample according to the measured beta particles or the measured Cherenkov radiation, where the determined condition includes a depth and/or a concentration of the radiotracers in the tissue sample.

### CROSS-REFERENCE TO RELATED APPLICATIONS

This application claims priority from U.S. Provisional Patent Application 61/342,321 filed Apr. 12, 2010, which is incorporated herein by reference.

### STATEMENT OF GOVERNMENT SPONSORED SUPPORT

This invention was made with Government support under contract CA133474 awarded by National Institutes of Health, and under contract 0854492 awarded by National Science Foundation. The Government has certain rights in this invention.

### FIELD OF THE INVENTION

The invention relates to tissue characterization. More specifically, the invention relates to methods for tissue characterization based on beta and Cherenkov radiation of molecular-specific radiotracers.

### BACKGROUND OF THE INVENTION

Surgical excision of diseased tissue, such as malignant tumors, requires the surgeon to identify abnormal tissue based on physical appearance. Although effective in the bulk tumor, the outer periphery is difficult to discriminate from the healthy tissue, due to the microscopic involvement. Thus, tissue samples are resected and sent to the pathologist to determine the existence of cancerous involvement. This process is laborious, and involves a significant amount of time, which comes at significant cost. Furthermore, this process involves a difficult trade-off between resecting too little, at a risk of not extracting all the tumor tissue, or resecting too much, causing unnecessary harm to the patient. The cost of tumor recurrence is great, and in most cases, the margin is extended by a few millimeters to ensure complete resection. In rectal cancer, it has been shown that margins <=1 mm result in a 6.5× greater chance of cancer recurrence vs. margins >2 mm. In breast cancer, it has been recommended that even if the margins have no involvement of cancer, further intervention is needed, such as by surgical or radiotherapeutic means. Thus, a fast, specific, method to determine cancer involvement is greatly needed.

### SUMMARY OF THE INVENTION

To address the needs in the art, a method of characterizing a tissue sample is provided that includes injecting a tissue sample with radiotracers, where the radiotracers include beta-emitter radio tracers, where the beta-emitter radio tracers emit beta particles according to a decay of the beta-emitter radio tracers, and measuring the beta particles or a Cherenkov radiation from the beta particles in the tissue sample, and determining a condition of the radio tracers in the tissue sample according to the measured beta particles or the measured Cherenkov radiation, where the determined condition includes a depth and/or a concentration of the radiotracers in the tissue sample.

According to one aspect of the invention, the radiotracers are molecular-specific radiotracers.

In a further aspect of the invention, the tissue sample includes an in vivo tissue sample or an in vitro tissue sample.

According to another aspect of the invention, determined condition of the radiotracers in the tissue sample are determined by illuminating the tissue sample with a light source, determining an intrinsic absorption spectrum of the tissue sample using a light detector, an endoscope, or a tissue imager, and comparing the absorption spectrum of the tissue sample with a Cherenkov radiation spectrum at the surface of the tissue sample from the measured Cherenkov radiation, where a spectral difference between the absorption spectrum and the Cherenkov radiation spectrum determines the depth and the concentration of the radiotracers in the tissue sample.

In yet another aspect of the invention, the depth and the concentration of the radiotracers in the tissue sample are determined by measuring the beta particles using a beta detector, where the beta detector includes a scintillator and a light detector, where when the beta particle travels through the scintillator, the scintillator radiation is produced and measured by the light detector, where the depth where the beta particles emitted in the tissue sample is known, due to the limited depth penetration of the beta particles, which is a known distribution dependent on their energy. According to one aspect, the beta particle detection is preformed using a plastic scintillating optical fiber coupled with a camera, or using a scintillating film on the tissue sample and imaging with the camera.

### BRIEF DESCRIPTION OF THE DRAWINGS

FIG. 1 shows optical light transmission spectra for three samples of tissue, according to one embodiment of the invention.

FIG. 2 shows the emitted Cherenkov optical spectra both superficially and through 2 mm of tissue, according to one embodiment of the invention.

FIG. 3 shows the signal (y-axis) due to the detection of beta emitters, for different samples of tissue (x-axis), according to one embodiment of the invention.

FIG. 4 shows one possible application of the imaging method where the system is used to inspect excised tissue, according to one embodiment of the invention.

FIG. 5 shows one application of the imaging method where the system is used intraoperatively to guide the surgeon, according to one embodiment of the invention.

FIG. 6 shows one possible application of the imaging method where the system is incorporated into an endoscope, according to one embodiment of the invention.

FIGS. 7a-7b show radioactive Na-22 beta-emitter located at a depth of 2 mm and emitted Cherenkov radiation, according to one embodiment of the invention.

FIG. 8 shows a flow diagram of the method according to one embodiment of the invention.

### DETAILED DESCRIPTION

This invention is a method to image excised or superficial tissue with high resolution, high sensitivity, and high specificity, which combines the micrometer-resolution of optical imaging with the specificity of disease-specific radiotracers. This is accomplished by imaging the emitted beta and/or Cherenkov radiation, which provide depth-selective imaging, although this technique is not restricted to depth-selective imaging. The optical tissue characterization via Cherenkov radiation is used to determine disease status by the high resolution imaging of targeted radiotracers in tissue samples. This characterization is used for disease diagnosis, both in vivo and in vitro, which allows on-site delineation of surgical margins during disease resection.

Radiotracers that are beta emitters will emit beta particles as they decay. These charged particles will radiate light if they exceed the apparent speed of light in a medium. Both the emitted light, which has a dominant blue contribution, and the beta particles, which have short range, identify the depth and concentration of the radiotracer through the following methods.

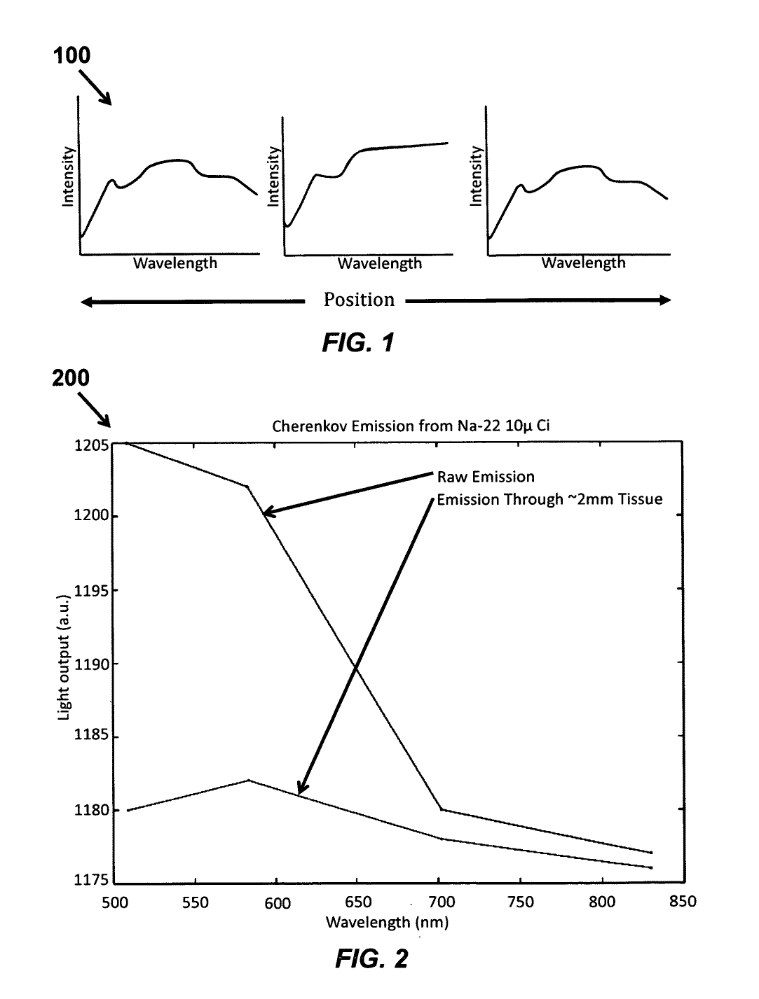

In one embodiment, depth and concentration of the radiotracers are determined by first illuminating the sample with optical light to detect the intrinsic absorption properties of the tissue. FIG. 1 shows three unique samples of the tissue spectra yield different spectra 100. This light can be imaged intraoperatively with a light detector, endoscopically with a fiber optic guidance, or separate from the surgeon as a standalone tissue imager, as discuss below and shown in FIGS. 4-6.

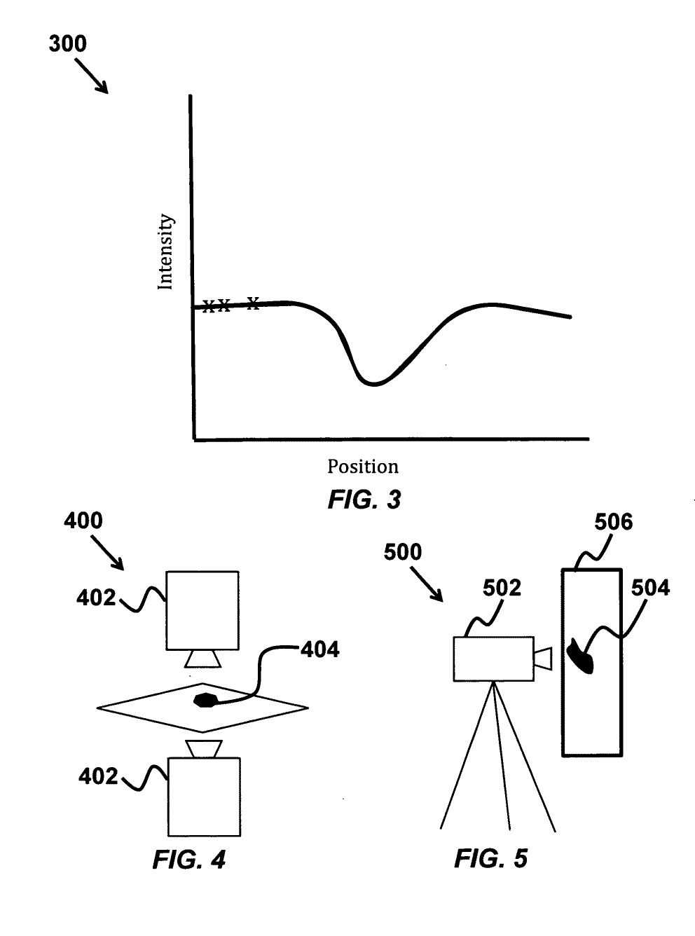

The Cherenkov spectrum exhibits spectral deformation based on absorption in tissue. FIG. 2 shows two spectrum 200: one spectrum is the Cherenkov radiation at the surface of the tissue sample, whereas the lower spectrum is due to the spectral shift due to light absorption through tissue. Because of the greater absorption of blue-green light, the peak of the Cherenkov radiation shift towards longer wavelengths. In another embodiment of this invention, the system uses the information provided by the Cherenkov absorption to determine depth and/or concentration. FIG. 3 shows an example of beta emission measurements 300 for samples located at different sites in the tissue.

In another application, the optical detector is operated independently of the surgery. This system measures imaging tissue specimens that have been excised from the tissue. These systems yield high-resolution images of tissue specimens, to determine margins. This invention has advantages over both optical and radio-guided imaging techniques, because it combines the advantages of each modality: the high specificity of radiotracers, with the superior resolution of optical imaging. The light detected from these systems determines a calibration for the tissue sample to determine depth and concentration. FIG. 4 shows an example external system 400, which is located outside the surgical area that has imagers 402 capable of imaging tissue specimens 404 excised from the tissue.

The radiotracer should be specific to the targeted disease such as any radioisotope which emits beta particles, e.g., 18F-FDG, Ga-68. Cherenkov radiation may be used in several ways to determine tissue abnormality. In one application, an optical detector could be mounted in the operating room or incorporated into an endoscope, so that it may examine the tissue during intervention. After injection of a radiotracer, the light detector is brought into close vicinity to the tissue, and an image is created upon which the surgeon might make a decision to excise tissue based on the image. FIG. 5 shows an example of one application, where the external system 500 may be mounted in the operating room, so that it may examine the tissue intraoperatively. After injection of a radiotracer, the camera 502 is brought into close vicinity to the tissue 504 in a patient 506 and an image is created upon which the surgeon might make a decision to excise tissue based on the image.

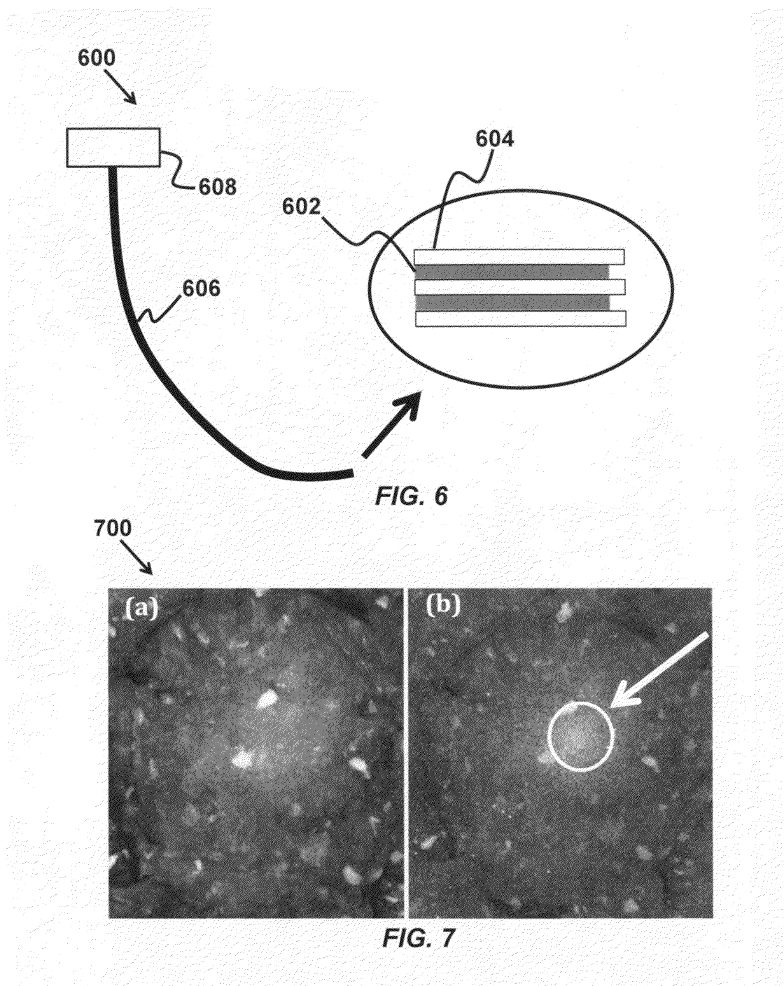

FIG. 6 shows an example of one application, where the system is incorporated into an endoscope 600. In one particular embodiment of this case, the shaded fibers are optical fibers 602 used to measure optical signal, while the white fibers are scintillating fibers 604 or optical fibers with scintillators on the end, that measure beta emission. In another embodiment, a beta detector 606 is incorporated in the system. Typical beta detectors are a plastic scintillator and a sensitive light detector. When a beta particle travels through a plastic scintillator, it produces a light signal distinct from the intrinsic Cherenkov signal.

Because the attenuation of beta radiation in tissue is well characterized, the beta scintillation provides additional information on the depth at which the betas are emitted. The beta detection could be performed either by using a plastic scintillating optical fiber, coupled to a sensitive camera, or by applying a thin scintillating film on the tissue sample and imaging directly with a sensitive camera. A system for the Cherenkov tissue characterization system could include three parts: the photoimaging system, the spectral discrimination system, and the radiotracer.

The photoimaging system could be built around maximizing resolution and light collection. This includes, but is not limited to, a microscope objective lens system, which is rasterized around the tissue, or a zoom lens, which images the entire specimen simultaneously. This may also include confocal optics for depth discrimination, or other types of optical systems.

The beta emission is imaged either by using a plastic scintillating optical fiber 606, coupled to a sensitive camera 608, or by applying a thin scintillating film on the tissue sample and imaging directly with a sensitive camera 608.

Spectral imaging should be built to maximize spectral information. This includes, but is not limited to, a filter wheel or tunable filter to image specific wavelengths, or a spectrograph for simultaneous wavelength collection.

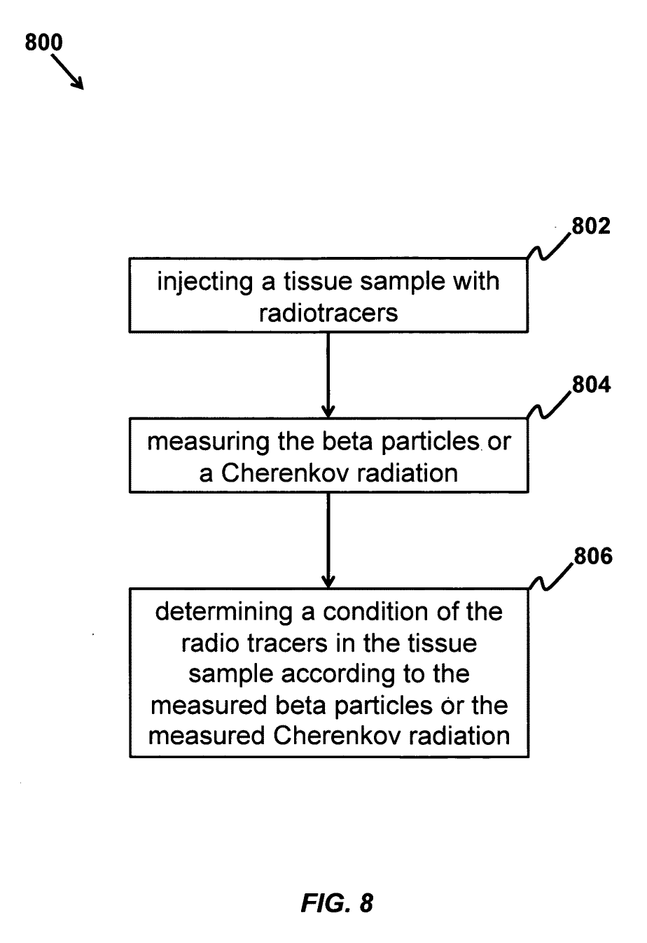

FIGS. 7a-7b show images 700 of a radioactive Na-22 beta-emitter located at a depth of 2 mm and emitted Cherenkov radiation, respectively.

FIG. 8 shows a flow diagram of the method 800 according to one embodiment of the invention. Here the method of characterizing a tissue sample is provided includes injecting a tissue sample with radiotracers 802, where the radiotracers include beta-emitter radiotracers, where the beta-emitter radiotracers emit beta particles according to a decay of the beta-emitter radiotracers, and measuring the beta particles or the Cherenkov radiation 804 from the beta particles in the tissue sample, and determining a condition of the radio tracers in the tissue sample according to the measured beta particles or the measured Cherenkov radiation 806, where the determined condition includes a depth and/or a concentration of the radiotracers in the tissue sample. Tissue with increased radiotracer concentration may be indicative of disease, so it is beneficial to know concentration. A preferred method to determine depth and concentration is by integrating a transparent scintillator into a portion of the distal end of the optical measurement device. Proximal to this, an optical filter is placed in front of the optical detector. The ratio between the light collected by the scintillator portion and the non-scintillating portion provides knowledge of depth. More specifically, the signal from the scintillator, S_{sc}, and the signal from the non-scintillating portion, S_{nsc}, of the light-collecting face, are collected. Because the optical signal from a single beta particle interacting with the scintillator may be 10,000 times greater than the Cerenkov signal, S_{sc} needs to be corrected for this signal difference. This could be done by an event-driven quantification (i.e., 10,000 scintillator photons equals 1 effective interaction, such that S_{sc}=1). A variable, κ, may be defined where:

$$κ = \frac{{S}_{sc}}{{S}_{nsc}}$$

If κ is larger than a threshold, τ, the device is within the range of beta emission. The range of the beta particles is well-known, and is based on the energy of the beta particles. Preferably, multiple measurements are taken until κ is maximum; this position indicates that the object is at the most probable range of betas. Since tissue optical properties in various tissues are known from literature, and depth is known from the range of the emitted betas, concentration can be calculated by correcting the Cerenkov emission signal for tissue absorption. This is typically done using Beer's law:

$$I = {I}_{0} {e}^{( - {μ}_{eff} * d )}$$

where I is intensity, μ_{eff}(λ) is the effective tissue absorption at a particular wavelength, λ, and d is the depth of the suspect lesion, which is determined based on κ. The concentration may be determined by correcting the Cerenkov intensity by Beer's law, or a similar light propagation theory, such as the diffusion approximation, that corrects for light attenuation in tissue. Additionally, pre-computed look-up tables may be used. The relationship between Cerenkov intensity and radiotracer concentration is well known as the Frank-Tamm formula (first reported by Frank and Tamm: I. Frank and I. Tamm, Compt. Rend. Acad. Sci. URSS 14 (109), 1939.) To perform this measurement, two acquisitions are used. One acquisition records the signal at the scintillation-emission wavelength with the scintillator placed in contact with the tissue, collecting both the Cerenkov and beta signals. For the next acquisition, the scintillator is removed from the tissue, either by moving the scintillator or retracting the scintillator, so that its location is beyond the range of the beta particles; in this way, only the Cerenkov signal is recorded. Radiotracer concentration may be determined if κ>τ.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art.

All such variations are considered to be within the scope and spirit of the present invention as defined by the following claims and their legal equivalents.

What is claimed:

1. A method of characterizing a tissue sample, comprising:
a. injecting a tissue sample with radiotracers, wherein said radiotracers comprise beta-emitter radio tracers, wherein said beta-emitter radio tracers emit beta particles according to a decay of said beta-emitter radio tracers; and
b. measuring said beta particles or a Cherenkov radiation from said beta particles in said tissue sample;
c. determining a condition of said radio tracers in said tissue sample according to said measured beta particles or said measured Cherenkov radiation, wherein said determined condition comprises i) a depth, ii) a concentration, or i) and ii) of said radiotracers in said tissue sample.

2. The method according to claim 1, wherein said radiotracers are molecular-specific radiotracers.

3. The method according to claim 1, wherein said tissue sample comprises an in vivo tissue sample or an in vitro tissue sample.

4. The method according to claim 1, wherein determined condition of said radiotracers in said tissue sample are determined by:
a. illuminating said tissue sample with a light source;
b. determining an intrinsic absorption spectrum of said tissue sample using i) a light detector, ii) an endoscope, or iii) a tissue imager; and
c. comparing said absorption spectrum of said tissue sample with a Cherenkov radiation spectrum at the surface of said tissue sample from said measured Cherenkov radiation, wherein a spectral difference between said absorption spectrum and said Cherenkov radiation spectrum determines said depth and said concentration of said radiotracers in said tissue sample.

5. The method according to claim 1, wherein said depth and said concentration of said radiotracers in said tissue sample are determined by:
a. measuring said beta particles, using a beta detector, wherein said beta detector comprises a scintillator and a light detector, wherein when said beta particle travels through said scintillator, said scintillator radiation is produced and measured by said light detector, wherein the depth where said beta particles emitted in said tissue sample is determined.

6. The method according to claim 5, wherein said beta particle detection is preformed using a plastic scintillating optical fiber coupled with a camera, or using a scintillating film on said tissue sample and imaging with said camera.
